(19) United States
(12) Patent Application Publication
(10) Pub. No.: US 2017/0275674 A1
(43) Pub. Date: Sep. 28, 2017

(54) NEXT-GENERATION SEQUENCING QUALITY CONTROL MONITORING TOOL

(71) Applicant: LIFE TECHNOLOGIES CORPORATION, Carlsbad, CA (US)

(72) Inventors: Kara Norman, San Francisco, CA (US); Patty Chiang, Walnut Creek, CA (US); Mahjabeen Yucekul, Oakland, CA (US)

(21) Appl. No.: 15/509,239

(22) PCT Filed: Sep. 14, 2015
(86) PCT No.: PCT/US15/49989

§ 371 Date: Mar. 7, 2017

Related U.S. Application Data
(60) Provisional application No. 62/050,654, filed on Sep. 15, 2014.

(51) Int. Cl. C12Q 1/68; G06F 19/00

(52) U.S. Cl. CPC C12Q 1/686; C12Q 1/6874; C12Q 1/6806; C12Q 1/6809; G06F 19/00; C12Q 2563/179; C12Q 2525/191; C12Q 2531/113; C12Q 2565/518

(57) ABSTRACT

Systems and methods are used to provide an online tool for clinical laboratories to monitor Next-Generation Sequencing (NGS) workflow using Quality Control (QC) material that can be used for multiple assays. The tool utilizes a highly multiplexed QC with NGS assays that detect somatic mutations. The control provides a common QC material that can be used across laboratories with different NGS instrument platforms, assays and bioinformatics pipelines to test precision and detect analytical deviations that may arise from reagent and instrument variation.

### CROSS-REFERENCE TO RELATED APPLICATIONS

This application claims the benefit of U.S. Provisional Patent Application Ser. No. 62/050,654, filed Sep. 15, 2014, which disclosure is herein incorporated by reference in its entirety.

### FIELD

The present disclosure is directed toward a next-generation sequencing quality control monitoring tool, and in particular, an online tool for clinical laboratories to monitor the next-generation sequencing using oncology hotspot control.

### INTRODUCTION

Next-Generation Sequencing technology has expanded beyond research applications to deliver clinically actionable test results that can effectively inform medical decision making. The utilization of next-generation sequencing in clinical settings is driven by the comprehensive capacity for genomic analysis and the potential to consolidate single-gene diagnostic tests.

Currently, laboratories lack uniform guidance on applying the technical aspects of quality management for quality control, which are essential to ensure the analytic validity of test results.

Next generation sequencing technologies provide a means to generate a large amount of sequence data. The implementation of next-generation sequencing technology in a clinical laboratory environment is complex, requiring significant infrastructure and expertise in clinical, scientific, and informatics specialties. Quality Control of sequence data generated from these technologies is extremely important for meaningful downstream analysis.

What is needed is a highly efficient and fast processing next-generation sequencing quality control monitoring tool to handle the large volume of datasets.

### BRIEF DESCRIPTION OF THE DRAWINGS

The skilled artisan will understand that the drawings, described below, are for illustration purposes only. The drawings are not intended to limit the scope of the present teachings in any way.

FIG. 1 shows a list of biomarkers that a laboratory may be interested in but may select a limited number of genes of interest, effectively “masking” variants that are not specific to their assays.

FIG. 2 shows a Next-Generation Sequencing Quality Control Monitoring tool that includes a Quality Control Monitoring site with a dashboard on a top menu that provides status information about the assays, such as select, uploading and monitoring the data, upon which embodiments of the present teachings may be implemented.

FIG. 3 shows an example of steps of a two-step process, where a user specifies parameters of the assay that they wish to track, in accordance with various embodiments of the invention.

FIG. 4 shows an example of steps of a two-step process, where a user specifies parameters of the assay that they wish to track, in accordance with various embodiments of the invention.

FIG. 5 shows a page of how the user can review settings and make changes to their assay, in accordance with various embodiments of the invention.

FIG. 6 shows an example if the user wants to edit the assay of FIG. 5, in accordance with various embodiments of the invention.

FIG. 7 shows an example page for making gene selection change(s) of the assay in FIG. 6, in accordance with various embodiments of the invention.

FIG. 8 shows a Quality Control Monitoring page, in accordance with various embodiments of the invention.

FIG. 9 shows an uploaded results page, in accordance with various embodiments of the invention.

FIG. 10 shows an Assay Result Details page, in accordance with various embodiments of the invention.

FIG. 11 shows an online Next-Generation Sequencing Quality Control Monitoring tool that allows a user to analyze results of the Acrometrix™ Oncology Hotspot Control from two or more sequencing runs based on the vcf files uploaded into the tool, in accordance with various embodiments of the invention.

FIG. 12 shows a Result for test assay of all of the vcf files a user has uploaded for an assay, in accordance with various embodiments of the invention.

FIG. 13 shows a Variant Count Chart stacked bar chart that displays the number of variants detected in analyzed vcfs, in accordance with various embodiments of the invention.

FIG. 14 shows an analysis summary of the number of variants detected and average read depth in a table, wherein the average read depth is based on read depth of only the variants selected for the assay, in accordance with various embodiments of the invention.

FIG. 15 shows a chart in which variants are detected inconsistently in some runs but not detected in other runs, in accordance with various embodiments of the invention.

FIG. 16 shows a chart of variants that were selected for the assay but not detected in the vcf, in accordance with various embodiments of the invention.

FIG. 17 shows a Allelic frequency graph, Allelic Frequency (%) vs. Run index, that visualizes trends of allelic frequency in the number of runs, in accordance with various embodiments of the invention.

FIG. 18 shows a chart of lab information that includes the laboratory name, the laboratory Id and other information related to the laboratory, in accordance with various embodiments of the invention.

FIG. 19 shows a chart to view the other users, in accordance with various embodiments of the invention.

FIG. 20 shows a chart to invite users and set up their access level, in accordance with various embodiments of the invention.

FIG. 21 is a block diagram that illustrates a computer system, upon which embodiments of the present teachings may be implemented.

FIG. 22 is a schematic diagram showing a system for generating an assay, in accordance with various embodiments.

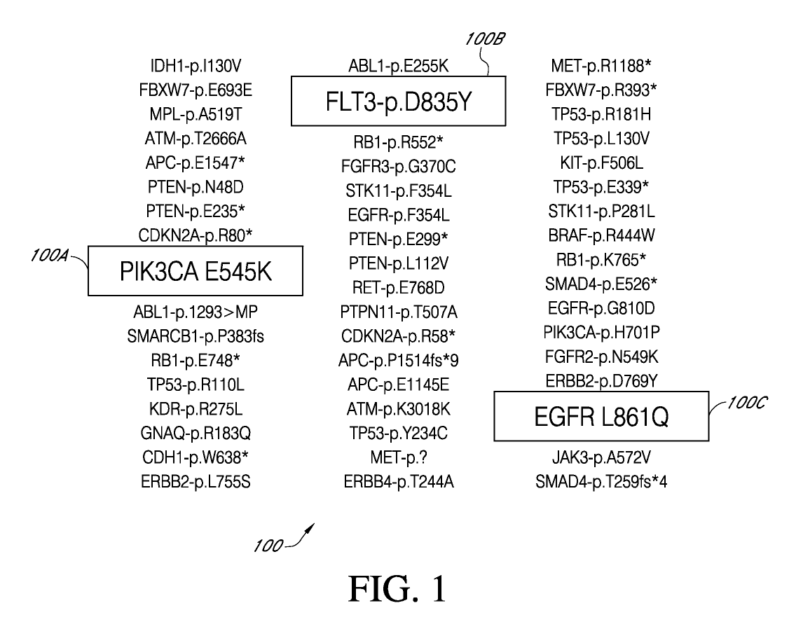

Before one or more embodiments of the present teachings are described in detail, one skilled in the art will appreciate that the present teachings are not limited in their application to the details of construction, the arrangements of components, and the arrangement of steps set forth in the following detailed description or illustrated in the drawings. Also, it is to be understood that the phraseology and terminology used herein is for the purpose of description and should not be regarded as limiting.

### EXEMPLARY EMBODIMENTS

The following description and the various embodiments described herein are exemplary and explanatory only and are not to be construed as limiting or restrictive in any way. Other embodiments, features, objects, and advantages of the present teachings will be apparent from the description and accompanying drawings, and from the claims.

Generally, clinical laboratories using next generation sequencing for somatic mutation detection have to either rotate through previously characterized samples, with unknown content and stability, or mix their own cell line pools, which is time-and resource-intensive.

The present invention solves many of these problems by providing an online tool for clinical laboratories to monitor Next-Generation Sequencing (NGS) workflow using routine Quality Control (QC) material that can be used for multiple assays, such as for example, the Acrometrix™ Oncology Hotspot Control (Benicia, Calif.). The tool utilizes a highly multiplexed QC with NGS assays that detect somatic mutations. The Oncology Hotspot Control provides a common QC material that can be used across laboratories with different NGS instrument platforms, assays and bioinformatics pipelines to test precision and detect analytical deviations that may arise from reagent and instrument variation. QC is an important part of a clinical laboratory's quality assurance program, and the availability of a platform-agnostic control will raise the standard for NGS in the clinical laboratory.

Oncology Hotspot Control uses an innovative proprietary high-multiplex blending process, and contains both synthetic and genomic DNA. In performance testing across multiple sites, for example, more than 30 external sites, the technology allows laboratories to standardize QC across multiple NGS platforms and refine their bioinformatic pipelines. For metrological traceability, the QC materials should be manufactured in cGMP facilities in accordance to ISO 13485 and follow ISO 17511:2003, which ensure lot-to-lot consistency of the control material. Currently, the Oncology Hotspot Control has a validated 18-month shelf life at −20° C. and is stable over five freeze-thaw events.

When the Oncology Hotspot Control is used in conjunction with the online NGS QC monitoring tool, a laboratory can monitor the performance of variants specific to its NGS assay. In one non-limiting example, the Oncology Hotspot Control allows users to control more than 500+ hotspot mutations with a single QC control and contains cancer-associated mutations across 53 genes, including KRAS, BRAF, ERBB2, EGFR and TP53. Users can choose from at least 500 single nucleotide variants, 19 insertions, 29 deletions, and three complex mutations. From the list of 500+ variants, the laboratory would select its variants of interest. The online NGS QC monitoring tool described herein would help the laboratory select and monitor those specific variants over multiple runs.

### Next-Generation Sequencing (NGS) Quality Control (QC) Monitoring Tool.

To get started, a user would log in to an online NGS QC Monitoring tool. The NGS QC Monitoring online tool has many pages and options for a user to select. Described below are some non-limiting embodiments and examples.

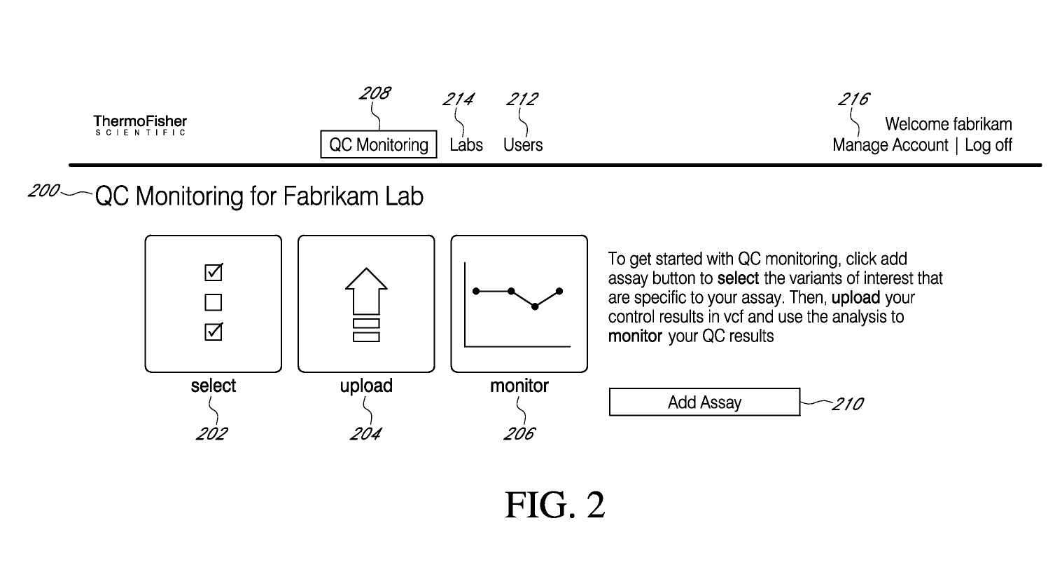

In one embodiment of an online NGS QC Monitoring tool shown in FIG. 2, the tool includes a QC Monitoring site 200 with a dashboard on a top menu that provides information about the assays, such as select 202 the variants of interest that are specific to the assay, upload 204 the control results in the vcf, and monitor the quality control results 206. To get back to this page from any other page on the site, click “QC Monitoring” 208 in the top menu.

The embodiment in FIG. 2 shows the dashboard before any assay information has been added. To start, the user clicks the “Add Assay” button 210 to enter information for the NGS assay the user wants to monitor using the Oncology Hotspot Control.

### Add Assay

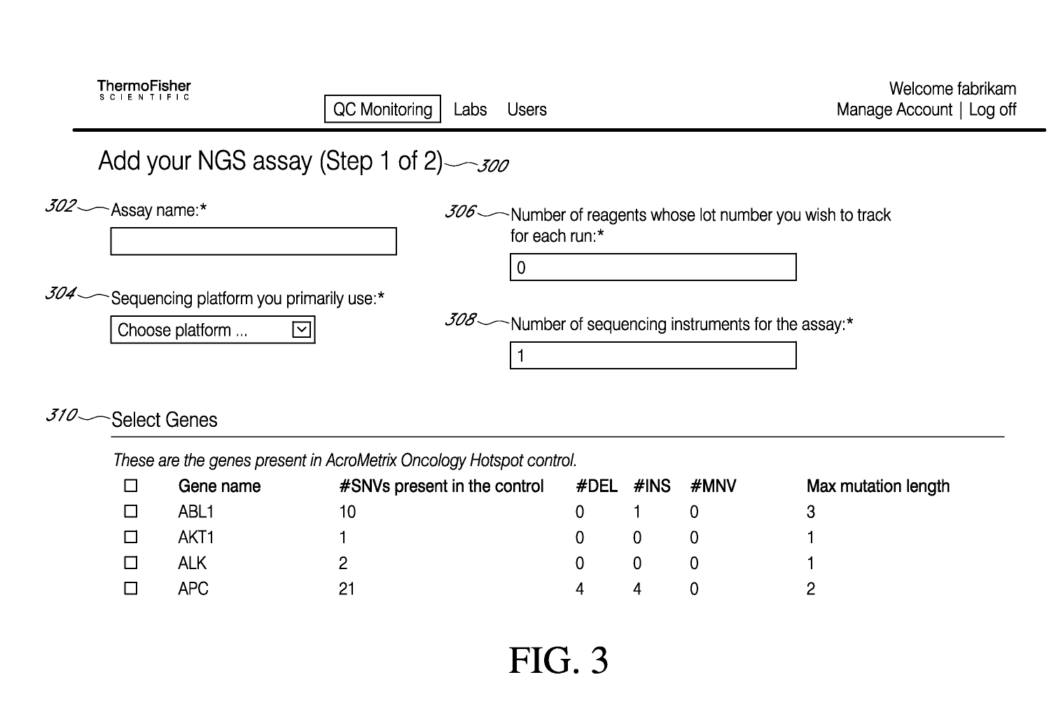

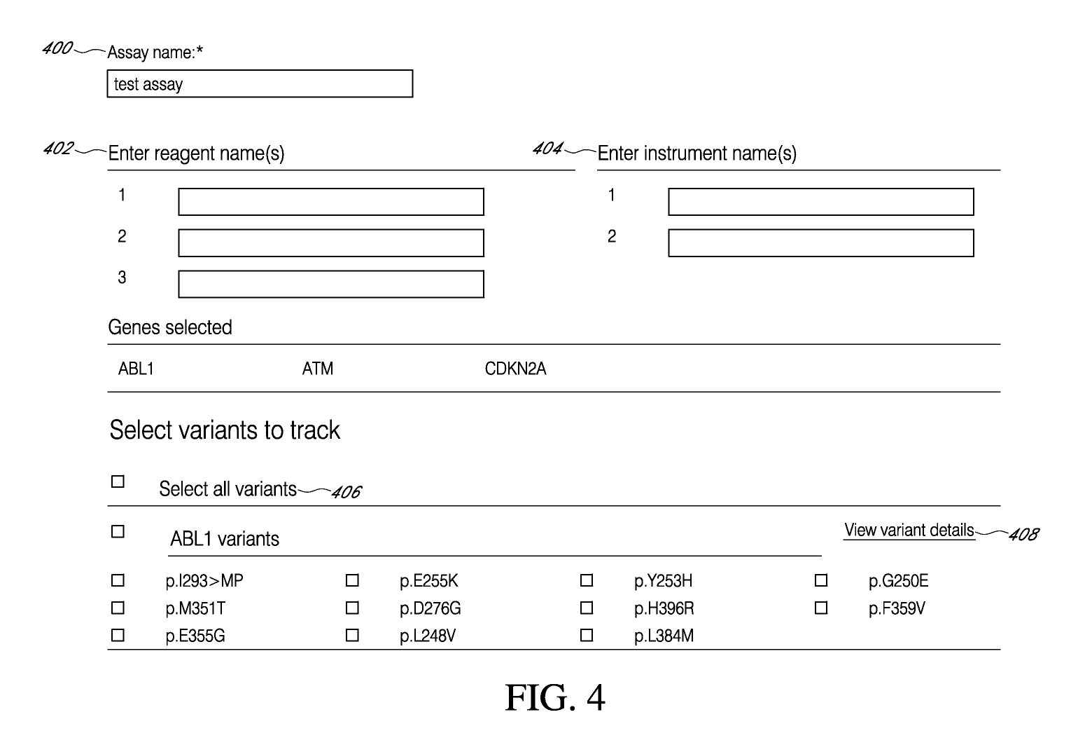

The “Add Assay” takes the user to the add screens. In the example shown, adding an NGS assay may be a two-step process (FIGS. 3 and 4), where the user specifies the parameters of the assay that they wish to track: such as the instruments used, the reagents whose lot numbers they wish to track, and the variants targeted by the panel.

In Step 1 of 2, shown in FIG. 3 the user enters the name of the assay 302 and the sequencing platform 304 they would like to use. The user can also specify the number of instruments if the lab has multiple sequencing instruments 308. In some examples, these inputs may be for records only and do not influence the analysis of results. Some laboratories track reagent lot numbers 306 in order to have a record to go back to when troubleshooting. The user may specify the number of reagents they wish to keep track of for each run. The user selects the genes they want to monitor 310. Presumably, the targeted variants are located on these genes.

In Step 2 of 2, shown in FIG. 4, the user enters the names of the reagents 402 and instruments 404 they are tracking. The instrument name can be its serial number or some other identifier used by operators in the selected lab.

Next, the user selects the variants they wish to track 406. The variants presented on this screen depend on the genes 310 the user selected in the previous step. By clicking on “View variant details” 408, the user can view COSMIC ID, mutation coding sequence, mutation type, and the length of mutation (if insertion or deletion) of the variants. When the user is finished with their inputs, they may click “Save” at the bottom of the page.

### Edit Assay

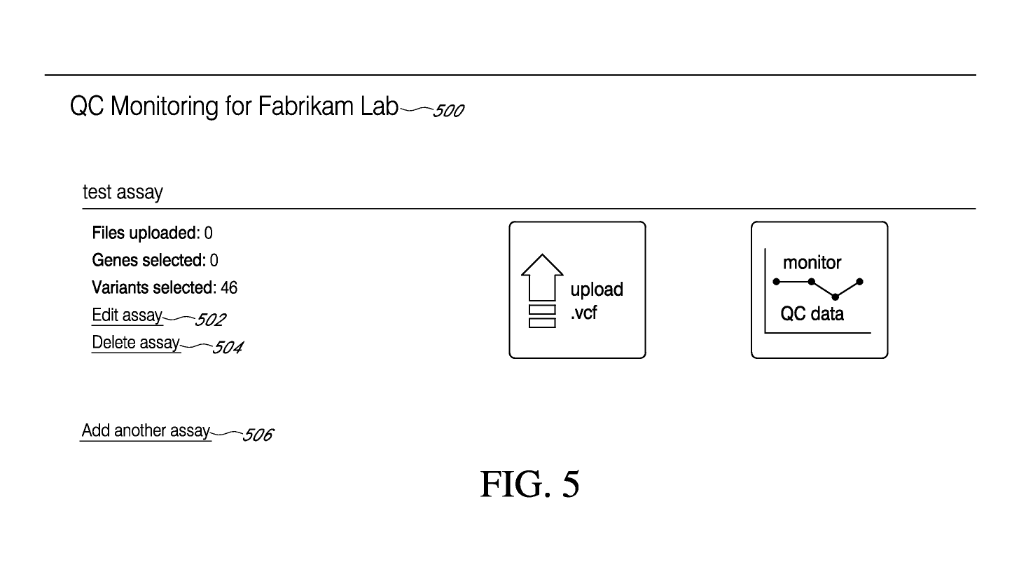

As shown in FIG. 5, the user can review settings and make changes to their assay by clicking “Edit assay” link 502 on the QC Monitoring dashboard. To change the variant selection, toggle the check box for the variant in the list then click “Save.” The user may also delete the assay 504 or may also add another assay 506, if needed.

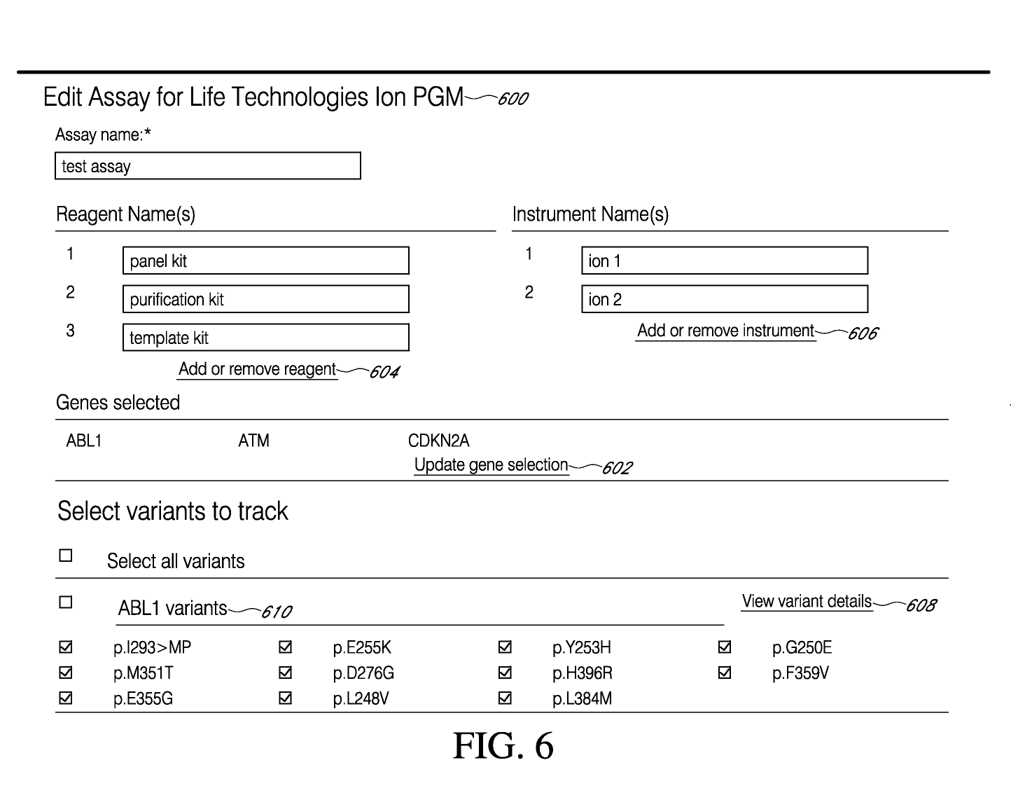

FIG. 6 shows an example if the user wants to edit the assay 600. The user may need to change the gene selection if the new variant is not on a gene that was previously selected. To change gene selection, click “Update gene selection” 602 in the Genes selected section of the page. The user may also add or remove reagents 604, add or remove instruments 606, view variant details 608 and change variants 610, if needed or desired. On the Edit Assay page, the user can also change reagent 604 or instrument names 606.

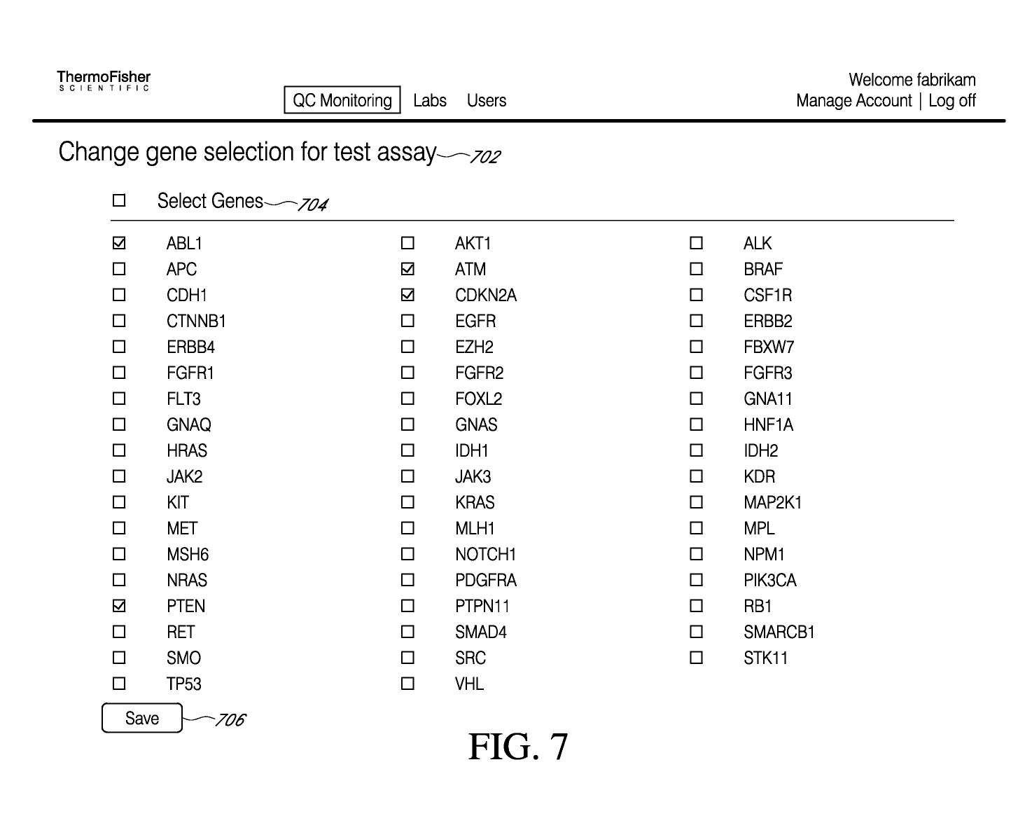

FIG. 7 shows an example page for making gene selection change 702. Once the user has checked/unchecked selected genes 704, the user clicks “Save” 706. This will take the user back to the Edit assay page (FIG. 6), where the user can select some or all of the variants 610 in newly selected genes.

### Upload Variant File

The online tool aggregates variant call data from selected run results of the Oncology Hotspot Control by parsing data from the vcf (Variant Call Format) file format. QC Monitoring tool support vcf version 4.0 or higher.

When the user has completed the sequencing run of the Oncology Hotspot Control, they can extract the vcf from the sequencing workflow and have the file ready for upload.

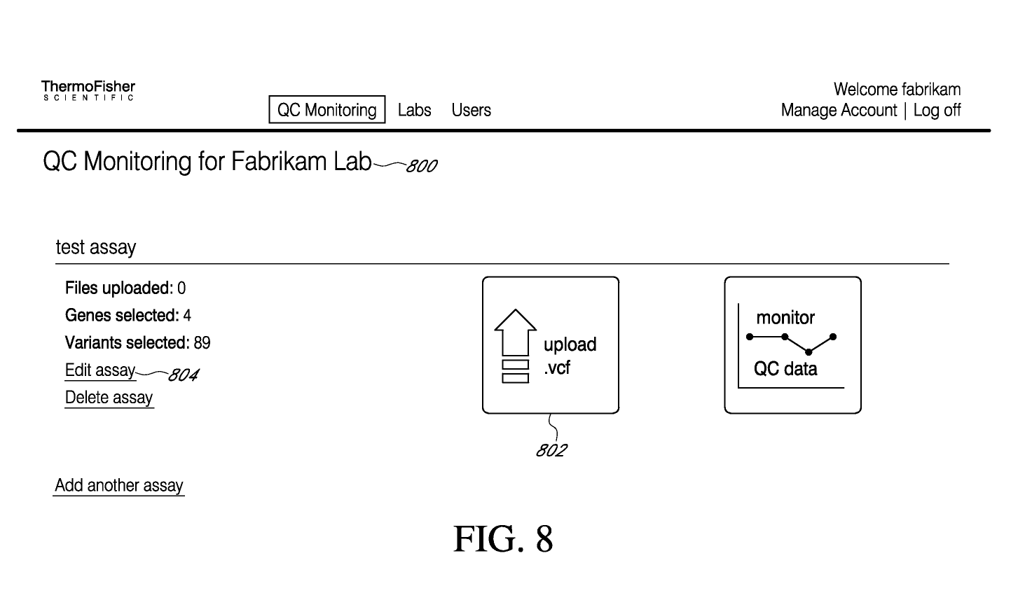

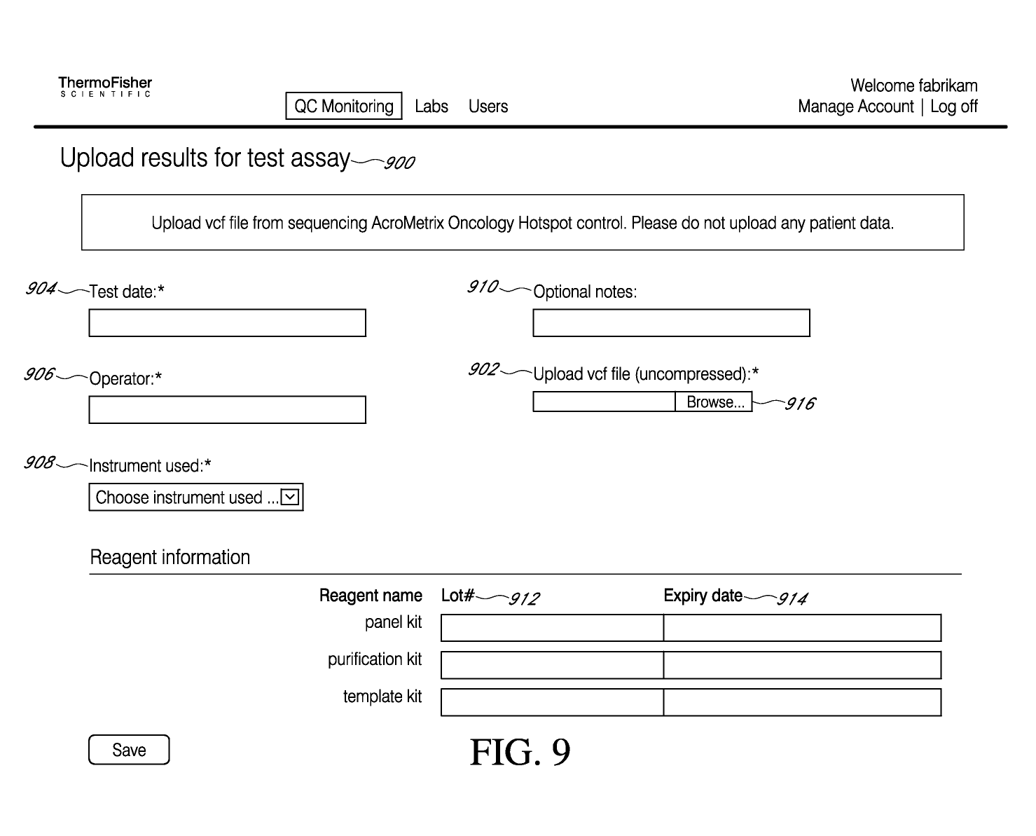

On the QC Monitoring page 800, shown in FIG. 8, the user clicks the “upload .vcf” button 802 to proceed. The “upload .vcf” button 802 takes the user to the upload results page 900, shown in FIG. 9, which collects run-specific information, uploads the vcf 902 and advises the successful upload of the vcf. The user enters information that is specific to the vcf about to uploaded: the date that the sequencing run was completed 904, the laboratory operator or technician who performed the test 906, the instrument used 908, and any additional notes 910. The reagent lot number 912 and expiry date 914 for the reagents used for this run may also be collected and tracked. The user may go back to “Edit Assay” Section 804 in FIG. 8 if they need to change the fields for the reagents or instruments. To upload the vcf, the user clicks “Browse” 916 and follows the on screen prompts. The tool parses the vcf.

In some embodiments, the screen may show an indication if the file upload and parsing is successful or if it fails. For example, in one embodiment, the screen may show a green ribbon at top if the file upload and parsing is successful, or a red ribbon at the top if the file upload or parsing fails.

.If upload fails, the user may check the validity of the vcf against the following criteria:
It must be in vcf version, for example, 4.0 or higher version format.
It must contain read depth and allelic frequency information. Allelic frequency can be directly present in the file or can be computed from alternate allele count.

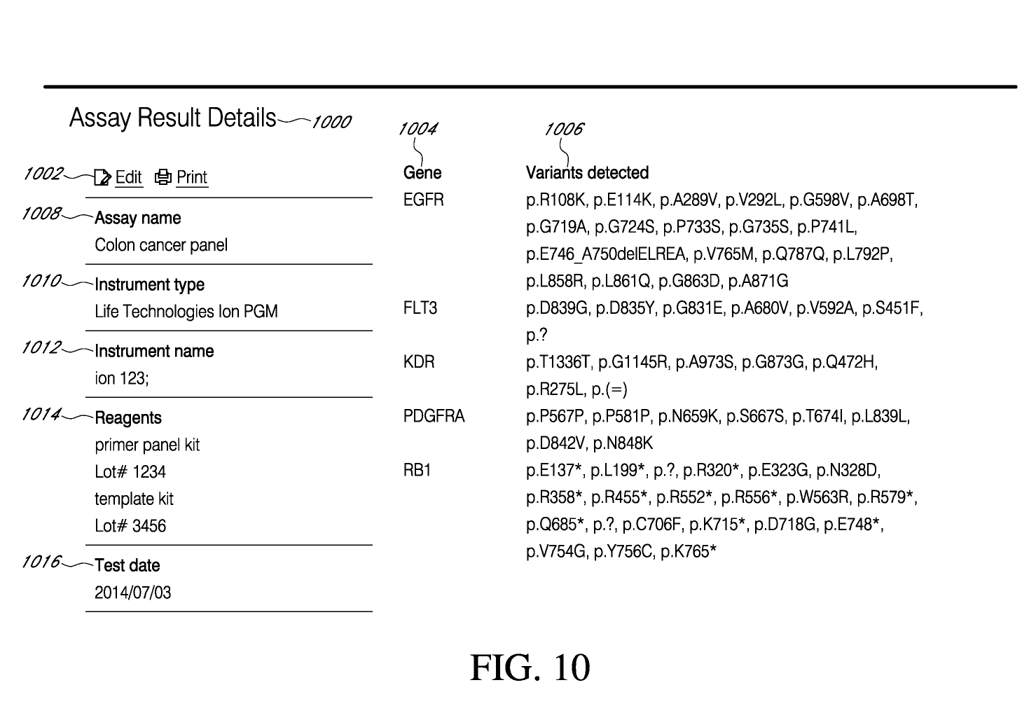

Upon a successful upload, the user can view the results of parsing the file by returning to the QC Monitoring dashboard shown in FIG. 2 and clicking on the “monitor QC data” button 208. By opening the “View” link for the respective vcf, the user can view an Assay Result Details page 1000, similar to the example shown in FIG. 10.

Assay information in the left column can be changed by clicking the “Edit” link. 1002. Variant information in the middle 1004 and right columns 1006 may be extracted from the uploaded vcf and may be changed by uploading a different vcf. Returning to the Results page by clicking the “monitor QC data” button on the QC Monitoring tab, click “Delete” to remove the vcf, and complete upload steps with the correct vcf file. Also included in the Assay Results Details may include the assay name 1008, the instrument type 1010, instrument name 1012, reagents 1014 and test date 1016.

### Analyze Results

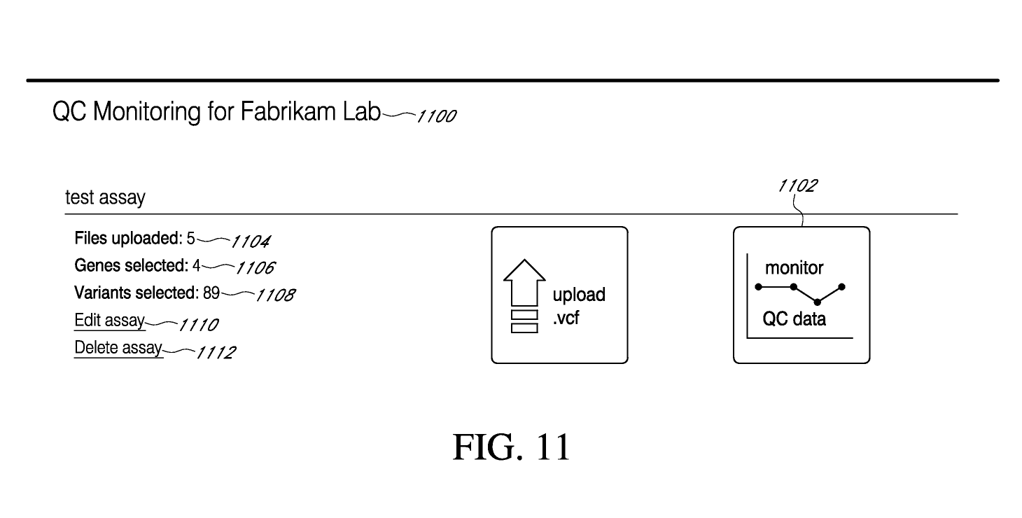

The online NGS QC Monitoring tool 1100 allows the user to analyze the results of the Oncology Hotspot Control from two or more sequencing runs based on the vcf files uploaded into the tool, such as shown in FIG. 11, which shows the test assay number of files uploaded 1104, genes selected 1106 and variants selected 1108. Click the “monitor QC data” button 1102 to begin analysis. The assay may also be edited 1110 or deleted 1112 from this page.

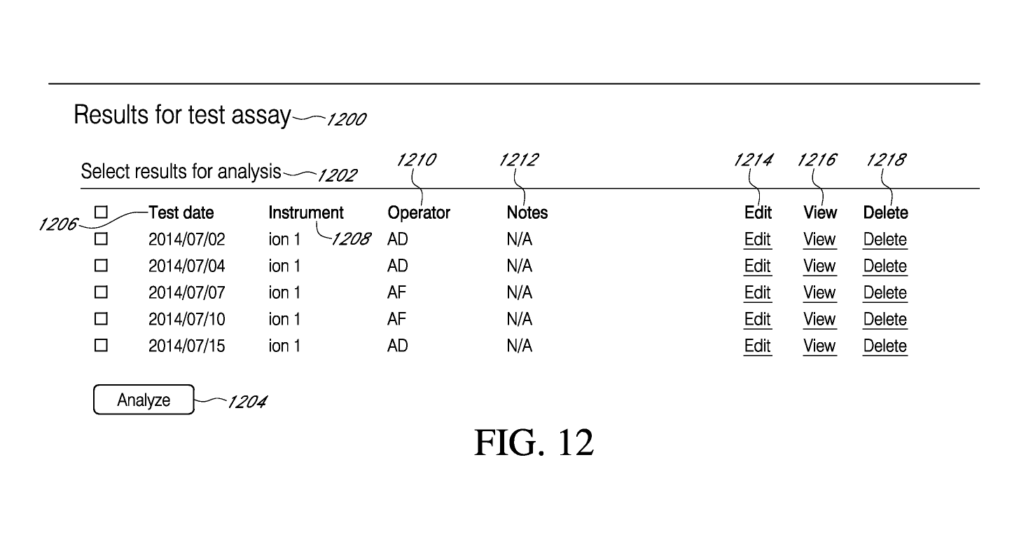

The Result Analysis page 1200 shown in FIG. 12 shows all of the vcf files 1202 the user has uploaded for this example assay. The user selects the vcf files they want to include in the analysis and then clicks the “Analyze” button 1204. This page may also show the test date 1206, instrument 1208, operator 1210, notes 1212 and may edit 1214, view 1216 or delete 1218 the select results.

The Result Analysis summarizes the results of the selected vcf files on a single page so that it can be printed or saved for the user quality records. The user can print this page directly from the browser. Additionally, a “Download” button at the bottom of the page allows the user to download the data for analysis using an analysis program, such as an excel file.

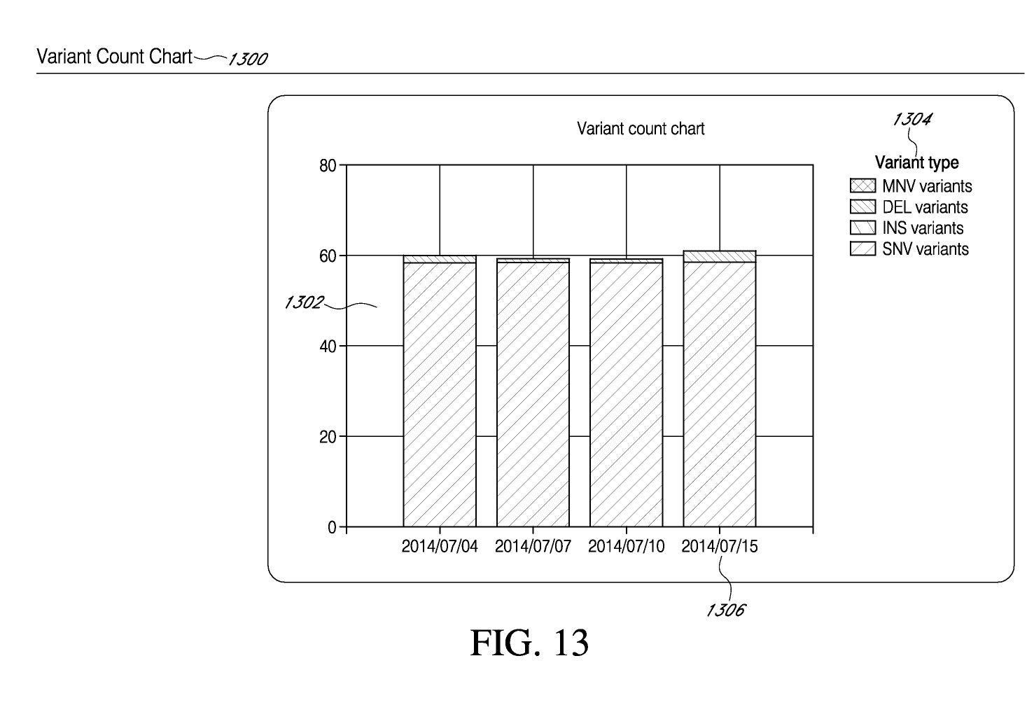

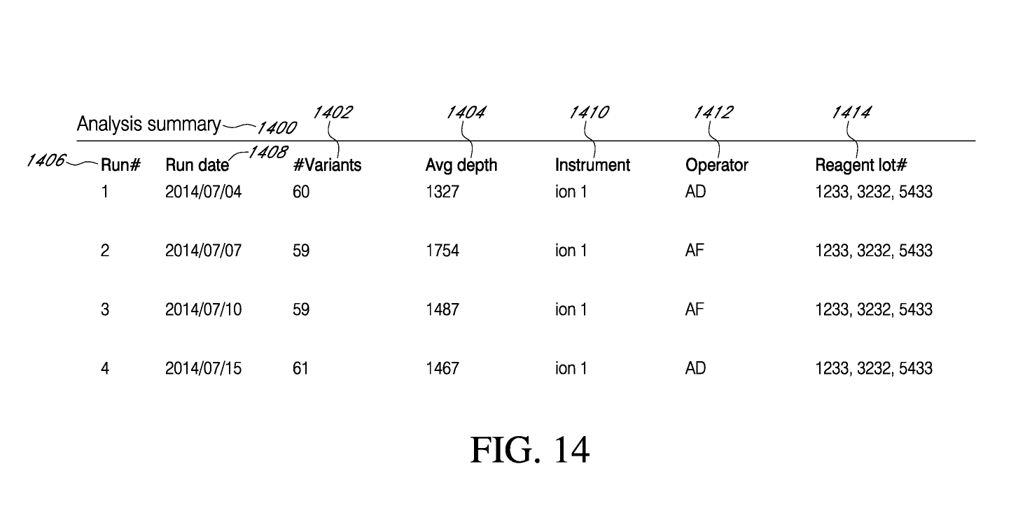

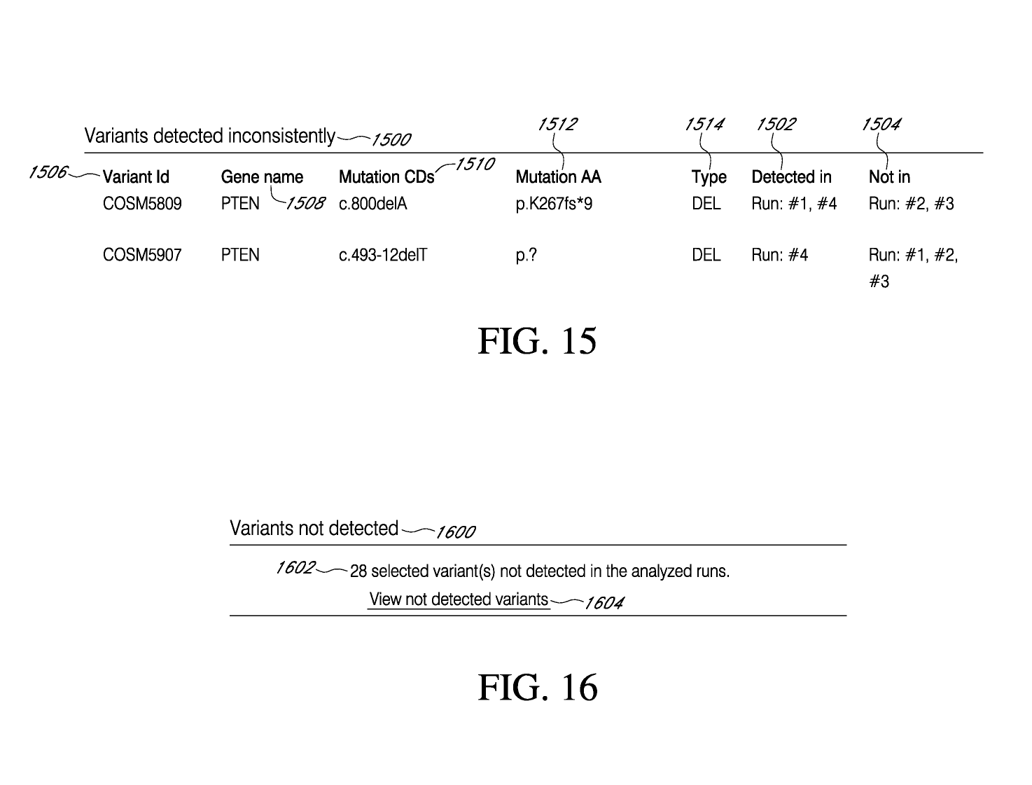

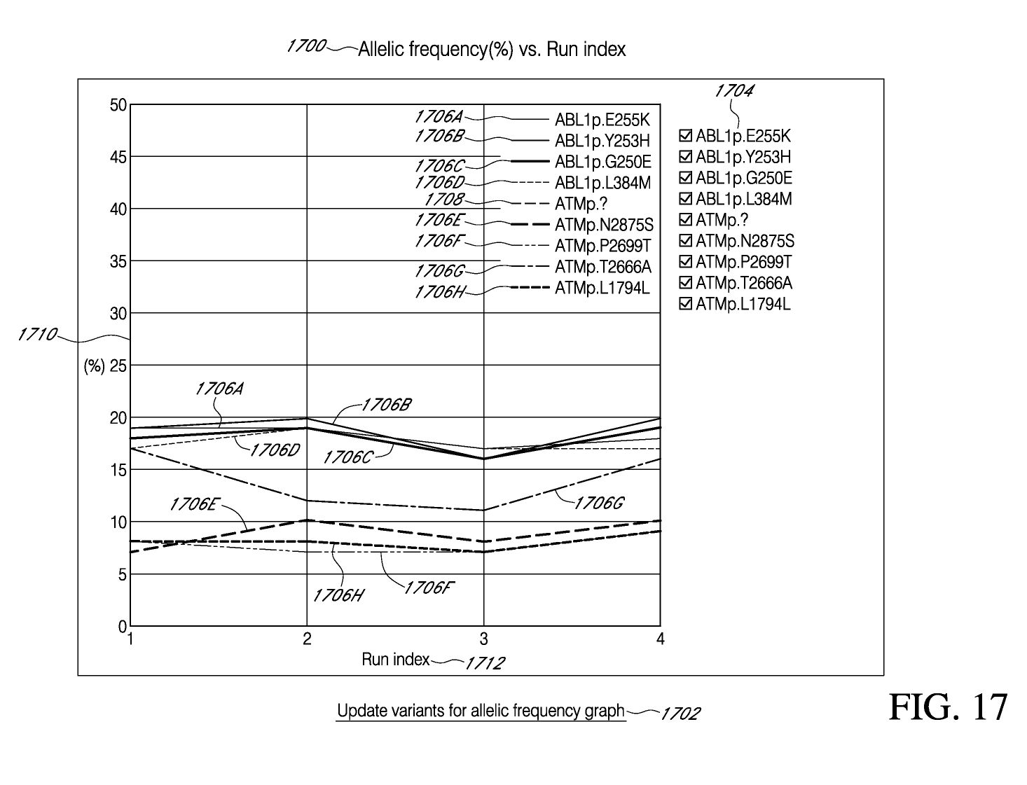

The Results Analysis may have multiple sections. In the examples shown, the Results Analysis has five sections:
1. Variant Count Chart (FIG. 13)
2. Analysis Summary (FIG. 14)
3. Variants Detected Inconsistently (FIG. 15)
4. Variants Not Detected (FIG. 16)
5. Allelic Frequency (%) vs. Run index (FIG. 17)

Variant Count Chart 1300 (FIG. 13)—Variant Count Chart is a stacked bar chart 1302 that displays the number of variants detected in the analyzed vcfs. The variants detected analysis is based on the list of variants 1304 the user selected when they set up the assay. The chart may also compare them on selected dates 1306.

Analysis Summary 1400 (FIG. 14)—Analysis summary presents the number of variants detected 1402 and average read depth 1404 in a table. The average read depth is based on read depth of only the variants selected for the assay. The chart may also show other information, such as the run number 1406, run date 1408, instrument 1410, operator 1412 and reagent lot number 1414.

Variants Detected Inconsistently 1500 (FIG. 15)—Variants detected inconsistently that are detected in some runs 1502 but not detected in other runs 1504 are listed in this section. The chart may also show other useful information such as the Variant Id 1506, Gene name 1508, Mutations CDS 1510, Mutations AA 1512 and Type 1514.

Variants Not Detected 1600 (FIG. 16)—If a variant is targeted by the user assay (and has been selected for in the Add Assay step) but is not detected in the vcf 1602, the NGS QC Monitoring tool displays the variants not detected in this table. Click “View not detected variants” 1604 to view these variants. The variants not detected page allows the user to remove variants from further tracking.

Allelic Frequency (%) vs. Run index 1700 (FIG. 17)—The Allelic frequency graph visualizes trends of allelic frequency 1710 in the number of runs 1712. In this example, there were four runs using nine variants 1706, for example, 1706A-1706H and one of the variants 1708 was not detected. In some graphs, up to 40 variants may be selected, viewing 20 at a time. To select the variants to view and compare, click the “Update variants for allelic frequency graph” link 1702. The allelic graph is interactive, enabling the user to turn on/off the visualization of the variants in the right hand list 1704. Use the “Update variants for allelic frequency graph” link to add or change the variants that can be added to this graph.

### Managing Lab Information

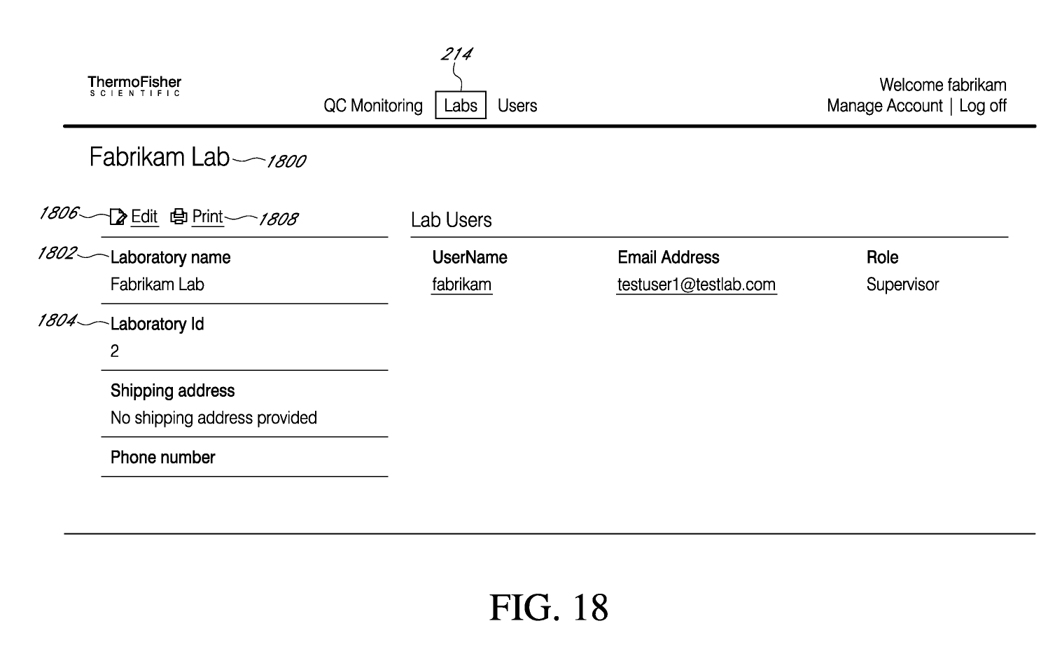

To review the lab information 1800, the user can click the Labs menu item 214, shown in FIG. 18. The lab information includes the laboratory name 1802, the laboratory Id 1804 and other information related to the laboratory. If the user needs to make changes, the user may click the “Edit” link 1804 on the page. The edit laboratory information page allows the user to make changes to the name and the shipping and contact information of the laboratory. The user may also print the information by pressing the print icon 1808.

### Managing User Information

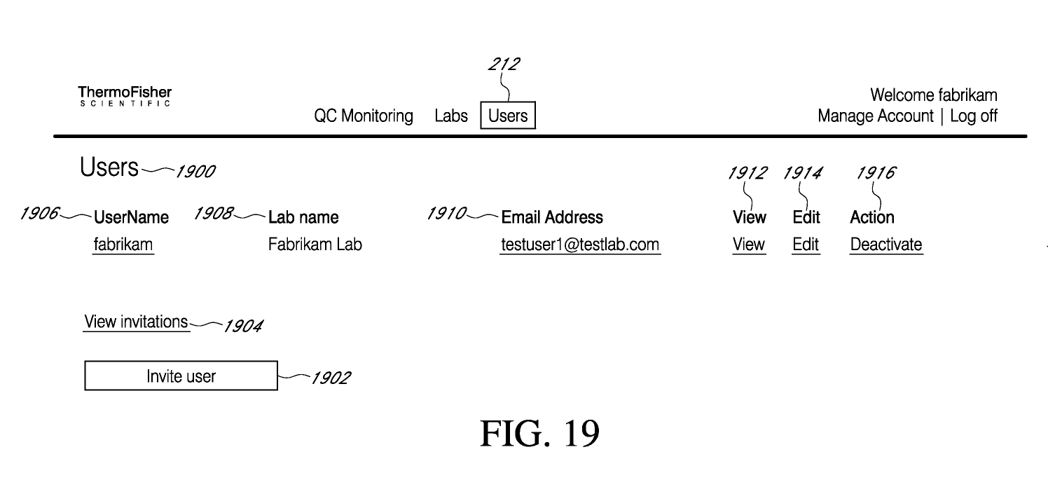

The user can view the other users 1900 in their lab, shown in FIG. 19, invite more users and activate/deactivate users by clicking the “Users” tab 212 on the top menu. From this page the user can also update their information. To add new users to their lab, the users invite other users 1902 to create an account by selecting the “Users” tab 212 from the top menu and clicking the “Invite user” button 1902. Other information on this page may include the View invitations, 1904, User name 1906, Lab name 1908, Email address 1910, view 1912, edit 1914 and action 1916 for each user.

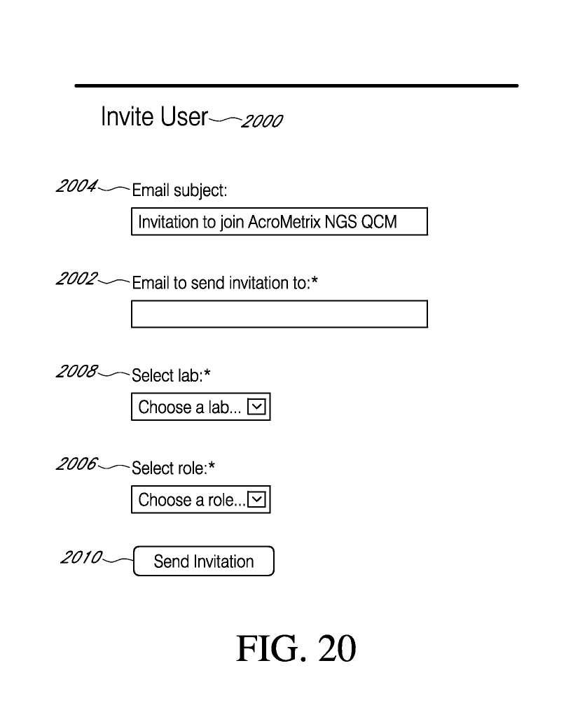

On the Invite User 2000 page, shown in FIG. 20, the user enters the new user's email address 2002 and the subject 2004. To set the new user's access level, the user selects the new user's role 2006. In some embodiments, the new user may be selected to be a “Supervisor” and “Operator”. Both “Supervisor” and “Operator” can upload results and perform analyses, but only “Supervisor” can perform user management tasks and change lab information. The new user may also be invited to select labs 2008. The user then clicks the “Send Invitation” button 2010 to invite the new user.

### Managing Account Information

A user may update their account information when they are logged in. A “Manage Account” link 216 is displayed in the top right corner of the page (see FIG. 2). Click on the link to update their information. Click “Save” when they are done updating. A user may also change their password on the “Manage Account” page, click “Change Password” link to go to the screen for password update. If the user forgets their password, they can use the reset password functionality on the login screen to automatically generate a temporary password for them. This will be sent to the email registered to their account. The user can login with the password and the change it to something the user can easily remember by going to “Manage Account” and then “Change Password” link. Once the user clicks “Forgot Password” on the login page, the reset password request form is presented. After filling in the information, click the button. The reset will proceed only if the information provided on this page matches the saved information for the user. They will receive an email with the newly generated password.

### Computer-Implemented System

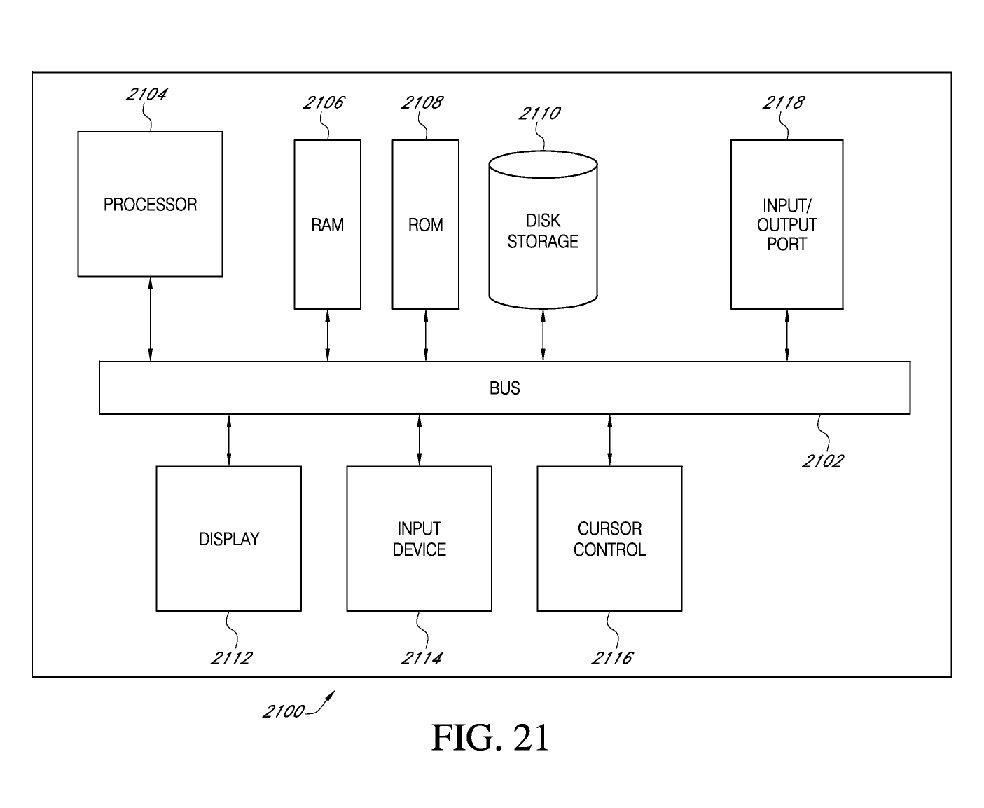

FIG. 21 is a block diagram that illustrates a computer system 2100, upon which embodiments of the present teachings may be implemented. Computer system 2100 includes a bus 2102 or other communication mechanism for communicating information, and a processor 2104 coupled with bus 2102 for processing information. Computer system 2100 also includes a memory 2106, which can be a random access memory (RAM) or other dynamic storage device, coupled to bus 2102 for determining base calls, and instructions to be executed by processor 2104. Memory 2106 also may be used for storing temporary variables or other intermediate information during execution of instructions to be executed by processor 2104. Computer system 2100 further includes a read only memory (ROM) 2108 or other static storage device coupled to bus 2102 for storing static information and instructions for processor 2104. A storage device 2110, such as a magnetic disk or optical disk, is provided and coupled to bus 2102 for storing information and instructions.

Computer system 2100 may be coupled via bus 2102 to a display 2112, such as a light-emitting diode (LED), liquid crystal display (LCD), or cathode ray tube (CRT), for displaying information to a computer user. An input device 2114, including alphanumeric and other keys, is coupled to bus 2102 for communicating information and command selections to processor 2104. Another type of user input device is cursor control 2116, such as a mouse, a trackball or cursor direction keys for communicating direction information and command selections to processor 2104 and for controlling cursor movement on display 2112. This input device typically has two degrees of freedom in two axes, a first axis (i.e., x) and a second axis (i.e., y), that allows the device to specify positions in a plane. In various embodiments, functionality of the input device 2116 and the display 2112 can be combined, such as with a touch screen.

A computer system 2100 can perform the present teachings. Consistent with certain implementations of the present teachings, results are provided by computer system 2100 in response to processor 2104 executing one or more sequences of one or more instructions contained in memory 2106. Such instructions may be read into memory 2106 from another computer-readable medium, such as storage device 2110. Execution of the sequences of instructions contained in memory 2106 causes processor 2104 to perform the process described herein. Alternatively hard-wired circuitry may be used in place of or in combination with software instructions to implement the present teachings. Thus implementations of the present teachings are not limited to any specific combination of hardware circuitry and software.

Computer system 2100 also includes input/output port 2118. Input/output port 2118 can be used to connect to a communications device. A communications device can include a wired or wireless network interface device. A wired or wireless network interface device can be connected to a network that is private or public. An exemplary public network is the Internet, for example. A wired or wireless network interface device can be connected to the Internet through one or more computers of one or more Internet service providers (ISPs). Computer system 2100 can be part of a system that can include, but is not limited to, a distributed computing system, a Web-based system, a cloud computing system, a software as a service system (SAAS), or any combination thereof.

The term “non-transitory computer-readable medium” as used herein refers to any tangible and non-transitory media that participates in providing instructions to processor 2104 for execution. Such a medium may take many forms, including but not limited to, non-volatile media, volatile media, and transmission media. Non-volatile media includes, for example, optical or magnetic disks, such as storage device 2110. Volatile media includes dynamic memory, such as memory 2106. Transmission media includes coaxial cables, copper wire, and fiber optics, including the wires that comprise bus 2102.

Common forms of computer-readable media include, for example, a floppy disk, a flexible disk, hard disk, magnetic tape, or any other magnetic medium, a CD-ROM, any other optical medium, punch cards, papertape, any other physical medium with patterns of holes, a RAM, PROM, and EPROM, a FLASH-EPROM, any other memory chip or cartridge, or any other tangible and non-transitory medium from which a computer can read.

Various forms of computer readable media may be involved in carrying one or more sequences of one or more instructions to processor 2104 for execution. For example, the instructions may initially be carried on the magnetic disk of a remote computer. The remote computer can load the instructions into its dynamic memory and send the instructions over a network, such as a wireless network, a wired network, or a combination thereof. Input/output port 2118 can receive the data from the network and place the data on bus 2102. Bus 2102 carries the data to memory 2106, from which processor 2104 retrieves and executes the instructions. The instructions received by memory 2106 may optionally be stored on storage device 2110 either before or after execution by processor 2104.

In accordance with various embodiments, instructions configured to be executed by a processor to perform a method are stored on a tangible and non-transitory computer-readable medium. The computer-readable medium can be a device that stores digital information. For example, a computer-readable medium may include flash memory devices, compact disc read-only memory (CD-ROM) or other devices known in the art for storing software. The computer-readable medium is accessed by a processor suitable for executing instructions configured to be executed.

The following descriptions of various implementations of the present teachings have been presented for purposes of illustration and description. It is not exhaustive and does not limit the present teachings to the precise form disclosed. Modifications and variations are possible in light of the above teachings or may be acquired from practicing of the present teachings. Additionally, the described implementation includes software but the present teachings may be implemented as a combination of hardware and software or in hardware alone. The present teachings may be implemented with both object-oriented and non-object-oriented programming systems.

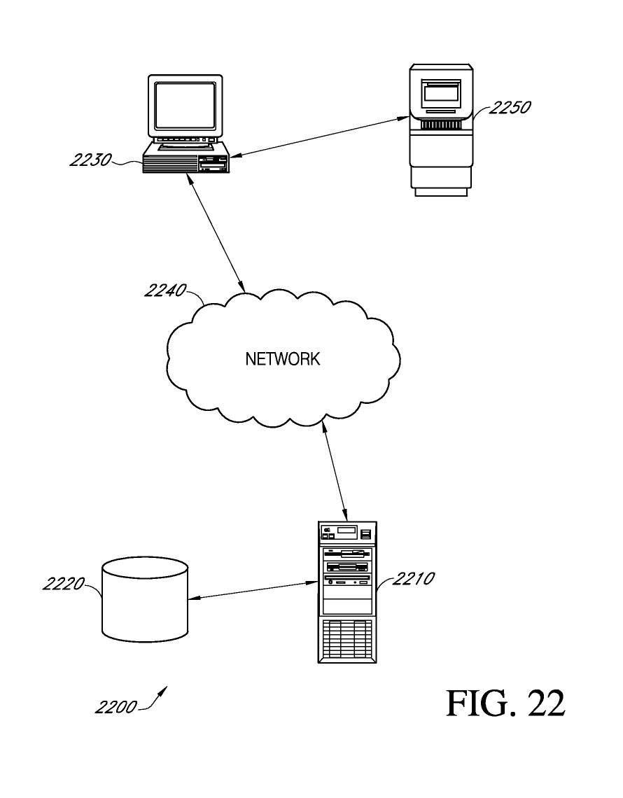

FIG. 22 is a schematic diagram showing a system 2200 for generating an assay, in accordance with various embodiments. System 2200 includes server computer 2210, database device 2220, and a communications device (not shown). Server computer 2210 is in communications with database device 2220 and the communications device. Database device 2220 is shown in FIG. 22 as connecting directly to server computer 2210. In various embodiments, database device 2220 can be connected indirectly to server computer 2210 through any private or public network including network 2240, for example.

Database device 2220 is shown in FIG. 22 as a device that is separate from server computer 2210. In various embodiments, database device 2220 can include a hardware component of server computer 2210, such a storage disk drive. Similarly, the communications device is, for example, a network interface device that is part of server computer 2210 in FIG. 22. In various embodiments, the communications device can be a device that is separate from server computer 2210.

Database device 2220 is shown in FIG. 22 as one physical device. One skilled in the art can appreciate that in various embodiments database device 2220 can include two or more physical devices. Database device 2220 can also include one or more logical databases.

Server computer 2210 receives an assay selection from client device 2230 of a laboratory through the communications device. For example, server computer 2210 is connected to network 2240 through the communications device. Client device 2230 is also connected to network 2240. As a result, server computer 2210 communicates with client device 2230 across network 2240. Network 2240 can be a private network or a public network. Network 2240 is, for example, the Internet. Server computer 2210 and client device 2230 can communicate across network 2240 using the hypertext transport protocol (HTTP), for example. Server computer 2210 and client device 2230 can then effectively communicate by exchanging Web pages, where server computer 2210 is a web server and client device 2230 is a Web client.

Client device 2230 may be connected to network 2240 through a client communications device. Client device 2230 can be, but is not limited to, a computer, a laboratory instrument, a tablet device, a mobile device, or any device capable of processing information and communicating across a network. Client device 2230 can also be connected directly to laboratory instrument 2250.

According to various embodiments, one or more features of any one or more of the above-discussed teachings and/or embodiments may be performed or implemented using appropriately configured and/or programmed hardware and/or software elements. Determining whether an embodiment is implemented using hardware and/or software elements may be based on any number of factors, such as desired computational rate, power levels, heat tolerances, processing cycle budget, input data rates, output data rates, memory resources, data bus speeds, etc., and other design or performance constraints.

Examples of hardware elements may include processors, microprocessors, input(s) and/or output(s) (I/O) device(s) (or peripherals) that are communicatively coupled via a local interface circuit, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth. The local interface may include, for example, one or more buses or other wired or wireless connections, controllers, buffers (caches), drivers, repeaters and receivers, etc., to allow appropriate communications between hardware components. A processor is a hardware device for executing software, particularly software stored in memory. The processor can be any custom made or commercially available processor, a central processing unit (CPU), an auxiliary processor among several processors associated with the computer, a semiconductor based microprocessor (e.g., in the form of a microchip or chip set), a macroprocessor, or generally any device for executing software instructions. A processor can also represent a distributed processing architecture. The I/O devices can include input devices, for example, a keyboard, a mouse, a scanner, a microphone, a touch screen, an interface for various medical devices and/or laboratory instruments, a bar code reader, a stylus, a laser reader, a radio-frequency device reader, etc. Furthermore, the I/O devices also can include output devices, for example, a printer, a bar code printer, a display, etc. Finally, the I/O devices further can include devices that communicate as both inputs and outputs, for example, a modulator/demodulator (modem; for accessing another device, system, or network), a radio frequency (RF) or other transceiver, a telephonic interface, a bridge, a router, etc.

Examples of software may include software components, programs, applications, computer programs, application programs, system programs, machine programs, operating system software, middleware, firmware, software modules, routines, subroutines, functions, methods, procedures, software interfaces, application program interfaces (API), instruction sets, computing code, computer code, code segments, computer code segments, words, values, symbols, or any combination thereof. A software in memory may include one or more separate programs, which may include ordered listings of executable instructions for implementing logical functions. The software in memory may include a system for identifying data streams in accordance with the present teachings and any suitable custom made or commercially available operating system (O/S), which may control the execution of other computer programs such as the system, and provides scheduling, input-output control, file and data management, memory management, communication control, etc.

According to various embodiments, one or more features of any one or more of the above-discussed teachings and/or embodiments may be performed or implemented using appropriately configured and/or programmed non-transitory machine-readable medium or article that may store an instruction or a set of instructions that, if executed by a machine, may cause the machine to perform a method and/or operations in accordance with the embodiments. Such a machine may include, for example, any suitable processing platform, computing platform, computing device, processing device, computing system, processing system, computer, processor, scientific or laboratory instrument, etc., and may be implemented using any suitable combination of hardware and/or software. The machine-readable medium or article may include, for example, any suitable type of memory unit, memory device, memory article, memory medium, storage device, storage article, storage medium and/or storage unit, for example, memory, removable or non-removable media, erasable or non-erasable media, writeable or re-writeable media, digital or analog media, hard disk, floppy disk, read-only memory compact disc (CD-ROM), recordable compact disc (CD-R), rewriteable compact disc (CD-RW), optical disk, magnetic media, magneto-optical media, removable memory cards or disks, various types of Digital Versatile Disc (DVD), a tape, a cassette, etc., including any medium suitable for use in a computer. Memory can include any one or a combination of volatile memory elements (e.g., random access memory (RAM, such as DRAM, SRAM, SDRAM, etc.)) and nonvolatile memory elements (e.g., ROM, EPROM, EEROM, Flash memory, hard drive, tape, CDROM, etc.). Moreover, memory can incorporate electronic, magnetic, optical, and/or other types of storage media. Memory can have a distributed architecture where various components are situated remote from one another, but are still accessed by the processor. The instructions may include any suitable type of code, such as source code, compiled code, interpreted code, executable code, static code, dynamic code, encrypted code, etc., implemented using any suitable high-level, low-level, object-oriented, visual, compiled and/or interpreted programming language.

According to various embodiments, one or more features of any one or more of the above-discussed teachings and/or embodiments may be performed or implemented at least partly using a distributed, clustered, remote, or cloud computing resource.

According to various embodiments, one or more features of any one or more of the above-discussed teachings and/or embodiments may be performed or implemented using a source program, executable program (object code), script, or any other entity comprising a set of instructions to be performed. When a source program, the program can be translated via a compiler, assembler, interpreter, etc., which may or may not be included within the memory, so as to operate properly in connection with the O/S. The instructions may be written using (a) an object oriented programming language, which has classes of data and methods, or (b) a procedural programming language, which has routines, subroutines, and/or functions, which may include, for example, C, C++, Pascal, Basic, Fortran, Cobol, Perl, Java, and Ada.

According to various embodiments, one or more of the above-discussed embodiments may include transmitting, displaying, storing, printing or outputting to a user interface device, a computer readable storage medium, a local computer system or a remote computer system, information related to any information, signal, data, and/or intermediate or final results that may have been generated, accessed, or used by such embodiments. Such transmitted, displayed, stored, printed or outputted information can take the form of searchable and/or filterable lists of runs and reports, pictures, tables, charts, graphs, spreadsheets, correlations, sequences, and combinations thereof, for example.

Various other embodiments may be derived by repeating, adding, or substituting any generically or specifically described features and/or components and/or substances and/or steps and/or operating conditions set forth in one or more of the above-described embodiments. Further, it should be understood that an order of steps or order for performing certain actions is immaterial so long as the objective of the steps or action remains achievable, unless specifically stated otherwise. Furthermore, two or more steps or actions can be conducted simultaneously so long as the objective of the steps or action remains achievable, unless specifically stated otherwise. Moreover, any one or more feature, component, aspect, step, or other characteristic mentioned in one of the above-discussed embodiments may be considered to be a potential optional feature, component, aspect, step, or other characteristic of any other of the above-discussed embodiments so long as the objective of such any other of the above-discussed embodiments remains achievable, unless specifically stated otherwise.

While the present teachings are described in conjunction with various embodiments, it is not intended that the present teachings be limited to such embodiments. On the contrary, the present teachings encompass various alternatives, modifications, and equivalents, as will be appreciated by those of skill in the art.

Further, in describing various embodiments, the specification may have presented a method and/or process as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process should not be limited to the performance of their steps in the order written, and one skilled in the art can readily appreciate that the sequences may be varied and still remain within the spirit and scope of the various embodiments.

1. A system used to provide a next-generation sequencing (NGS) quality control monitoring tool, comprising:
a database device configured to store quality control (QC) information associated with a multiplexed oncology hotspot control for use in multiple NGS assays that are performed by multiple NGS instruments; and
an oncology hotspot control server computer configured to:
receive a selection of multiple
NGS assays from each of a plurality of client devices, each of the plurality of client devices being associated with a respective NGS instrument from among the multiple NGS instruments;
transmit information associated with the selection of the multiple NGS assays to a respective NGS instrument that is in communication with a respective client device from among the plurality of client devices; and
receive, from the respective NGS instrument, an indication that an analytical deviation has occurred when performing one or more of the multiple NGS assays based upon the QC information stored in the database device,
wherein the QC information is utilized by each of the multiple NGS instruments when performing respective NGS assays.

2. The system of claim 1, wherein the client device is associated with a laboratory.

3. The system of claim 1, wherein the oncology hotspot control server computer is further configured to receive assay results from one or more of the plurality of client devices.

4. (canceled)

5. The system of claim 1, wherein the hotspot control server computer is further configured to receive, from one or more of the plurality of client devices, changes to one or more of the multiple NGS assays for use in running subsequent NGS assays.

6. A next-generation sequencing (NGS) quality control monitoring tool, comprising:
a database device configured to store quality control (QC) information associated with a multiplexed oncology hotspot control for use in multiple NGS assays;
multiple NGS instruments configured to utilize the QC information when performing multiple NGS assays; and
an oncology hotspot control server computer configured to detect analytical deviations of the multiple NGS assays between each of the multiple NGS instruments based upon the QC information stored in the database device to standardize the QC information across each of the multiple NGS instruments.

7. The tool of claim 6, further comprising:
a client device in communication with one or more of the multiple NGS instruments, the client device being configured to allow a user to select a plurality of mutations for the multiple NGS assays.

8. The tool of claim 6, further comprising:
a client device in communication with one or more of the multiple NGS instruments, the client device being configured to present a dashboard to facilitate selection of the multiple NGS assays.

9. The tool of claim 8, wherein the client device is further configured to present the dashboard to facilitate (i) a selection of variants of interest that are specific to the multiple NGS assays, (ii) uploading results of the multiple NGS assays in Variant Call Format (vcf) to the oncology hotspot control server computer, and (iii) monitoring the results of the multiple NGS assays.

10. The tool of claim 9, wherein the client device is further configured to present the dashboard to facilitate entry of parameters of the one or more of the multiple NGS assays, including (i) one or more of the multiple NGS instruments used to perform the multiple assays, (ii) names of the reagents used in the multiple NGS assays, (iii) a number of reagents to track one or more assay runs within the multiple NGS assays, (iv) variants targeted by a panel, and (v) genes to be monitored.

11. The tool of claim 10, wherein the parameters further include lot numbers of the reagents.

12. The tool of claim 8, wherein the client device is further configured to present the dashboard to facilitate (i) adding, removing, or changing reagents, (ii) adding, removing, or changing one or more of the multiple NGS instruments, (iii) viewing variant details, and (iv) changing variants.

13. The tool of claim 6, wherein the oncology hotspot control server computer is further configured to receive variant call data results from the multiple NGS assays in Variant Call Format (vcf), and to aggregate the variant call data results by parsing data in accordance with the vcf file format.

14. The tool of claim 13, further comprising:
a client device in communication with the oncology hotspot control server computer,
wherein the client device allows a user to analyze the results of the multiple NGS assays based on the vcf files uploaded to the oncology hotspot control server computer.

15. The tool of claim 6, further comprising:
a client device in communication with the oncology hotspot control server computer, the client device being configured to present details associated with results of the multiple NGS assays including an assay name, an instrument type, an instrument name, reagents and test dates.

16. A method of using a next-generation sequencing (NGS) multiplexed quality control, comprising:
providing at least one multiplexed oncology hotspot control for use in multiple NGS assays;
selecting the quality at least one multiplexed oncology hotspot control for use in multiple next-generation sequencing to be performed on multiple NGS instruments; and
detecting analytical deviations of the multiple NGS assays between the multiple NGS instruments based upon the at least one multiplexed oncology hotspot control to standardize the at least one multiplexed oncology hotspot control across the multiple NGS instruments.

17. The method of claim 16, further comprising:
displaying a dashboard to control which of the one or more of the multiple NGS assays are to be performed on the multiple NGS instruments.

18. The method of claim 17, further comprising:
receiving, via the dashboard, a selection of variants of interest that are specific to the multiple NGS assays;
uploading, via the dashboard, the results of the multiple NGS assays in Variant Call Format (vcf) to an oncology hotspot control server computer; and
monitoring, via the dashboard, the results of the multiple NGS assays.

19. A non-transitory, tangible computer-readable storage medium storing machine readable instructions on an oncology hotspot control server computer for a next-generation sequencing (NGS) quality control monitoring tool that, when executed on the oncology hotspot control server computer cause the oncology hotspot control server computer to:
store, in a database device, quality control (QC) information associated with a multiplexed oncology hotspot control for use in multiple NGS assays that are performed by multiple NGS instruments;
receive a selection of multiple NGS assays from each of a plurality of client devices, each of the plurality of client devices being associated with a respective NGS instrument from among the multiple NGS instruments;
transmit information associated with the selection of the multiple NGS assays to a respective NGS instrument that is in communication with a respective client device from among the plurality of client devices;
receive, from the respective NSG instrument, an indication that an analytical deviation has occurred when performing one or more of the multiple NSG assays based upon the QC information stored in the database device;
wherein the QC information is utilized by each of the multiple NSG instruments when performing respective NSG assays.

20. The non-transitory, tangible computer-readable medium of claim 19, wherein the non-transitory, tangible computer-readable medium further includes machine readable instructions that, when executed by the oncology hotspot control server computer, cause the oncology hotspot control server computer to receive assay results from one or more of the plurality of client devices.

21. The non-transitory, tangible computer-readable medium of claim 19, wherein the non-transitory, tangible computer-readable medium further includes machine readable instructions that, when executed by the oncology hotspot control server computer, cause the oncology hotspot control server computer to receive, from one or more of the plurality of client devices, changes to one or more of the multiple NGS assays for use in running subsequent NGS assays.
